(19) United States
(12) Patent Application Publication
(10) Pub. No.: US 2016/0121115 A1
(43) Pub. Date: May 5, 2016

(54) SYSTEMS AND METHODS FOR ELECTRICAL STIMULATION OF NEURAL TISSUE

(71) Applicant: RIPPLE LLC, Salt Lake City, UT (US)

(72) Inventors: Kenneth S. Guillory, Salt Lake City, UT (US); Scott Hiatt, Sandy, UT (US); Andrew Wilder, Salt Lake City, UT (US); Daniel McDonnall, Salt Lake City, UT (US); Christopher F. Smith, North Salt Lake, UT (US)

(21) Appl. No.: 14/891,624

(22) PCT Filed: May 15, 2014
(86) PCT No.: PCT/US14/38266

§ 371 Date: Nov. 16, 2015

Related U.S. Application Data
(60) Provisional application No. 61/823,398, filed on May 15, 2013.

(51) Int. Cl. A61N 1/36; A61N 1/378; A61N 1/372

(52) U.S. Cl. CPC A61N 1/36017; A61N 1/37217; A61N 1/3787

(57) ABSTRACT

Disclosed herein are various embodiments of electrical stimulation systems configured to stimulate tissue in a subject. The system may include a controller configured to send at least one stimulation pattern to be implemented by the electrical stimulation system. The controller may include a first digital control interface. The system may also include a stimulation module that includes a second digital control interface configured to be in electrical communication with the first digital control interface. The stimulation circuitry may be configured to implement the at least one stimulation pattern as an analog stimulation signal based on an ongoing stream of digital commands received from the controller. The system may further comprise a percutaneous connector assembly configured to be coupled to a subject through the subject's skin. The percutaneous connector may include a second connector configured to couple to the first connector and a first electrode lead.

### RELATED APPLICATIONS

The present application claims the benefit of Patent Cooperation Treaty Application No. PCT/US2014/038266, filed May 15, 2014 and titled “SYSTEMS AND METHODS FOR ELECTRICAL STIMULATION OF NEURAL TISSUE,” which claims the benefit of U.S. Patent Application 61/823,398, filed May 15, 2013 and titled “SYSTEMS AND METHODS FOR ELECTRICAL STIMULATION,” each of which is incorporated herein by reference in its entirety.

### TECHNICAL FIELD

This disclosure relates to systems and methods for electrically stimulating tissue for physiology research, prosthetic, and neuroprosthetic applications.

### BACKGROUND

In electrophysiological stimulation and recording applications, connection and cabling to stimulators can be bulky and cumbersome. Long cables between the stimulator circuitry and the percutaneous cable can also pick up more environmental noise. Further, motion of the cabling connected to a percutaneous connector and/or electrodes may create noise in signals that tend to be relatively weak. As such, even a relatively small amount of noise may significantly impact the signal to noise ratio of the signal.

### BRIEF DESCRIPTION OF THE DRAWINGS

Non-limiting and non-exhaustive embodiments of the disclosure are described, including various embodiments of the disclosure with reference to the figures, in which:

FIG. 1 illustrates a functional block diagram of a system for electrical stimulation of neural tissue consistent with embodiments disclosed herein.

FIG. 2 illustrates a functional block diagram of a system for electrical stimulation of neural tissue in which the connector assembly may be used to connect to electrodes temporarily while the stimulation module is held nearby to the subject.

FIG. 3 illustrates a functional block diagram of a system for electrical stimulation of neural tissue that includes a watchdog module, an error correction module, and a calibration module consistent with embodiments disclosed herein.

FIG. 4 illustrates a functional block diagram of a system for electrical stimulation of neural tissue that includes a plurality of sets of stimulation circuitry in the stimulation module consistent with embodiments disclosed herein.

FIG. 5 illustrates a functional block diagram of a system for electrical stimulation of neural tissue that includes a plurality of sensor components consistent with embodiments disclosed herein.

FIG. 6 illustrates a functional block diagram of a system for electrical stimulation of neural tissue that includes a recording module consistent with embodiments disclosed herein.

FIG. 7 illustrates a functional block diagram of a system for electrical stimulation of neural tissue that includes a stimulation module within an electrode connector assembly consistent with embodiments disclosed herein.

FIG. 8 illustrates a functional block diagram of a system for electrical stimulation of neural tissue that includes a wireless module consistent with embodiments disclosed herein.

FIG. 9 illustrates a functional block diagram of a system for electrical stimulation of neural tissue that includes a wireless module and an inductive power receiver consistent with embodiments disclosed herein.

A detailed description of systems and methods consistent with embodiments of the present disclosure is provided below. While several embodiments are described, it should be understood that the disclosure is not limited to any one embodiment, but instead encompasses numerous alternatives, modifications, and equivalents. In addition, while numerous specific details are set forth in the following description, in order to provide a thorough understanding of the embodiments disclosed herein, some embodiments can be practiced without some or all of these details. Moreover, for the purpose of clarity, certain technical material that is known in the related art has not been described in detail to avoid unnecessarily obscuring the disclosure.

### DETAILED DESCRIPTION

The inventors of the present disclosure have recognized that various advantages may be achieved in neural stimulation and recording systems by having stimulator and recording circuitry directly and mechanically coupled to a percutaneous connector assembly. Further, the inventors of the present disclosure have recognized that use of digital interfaces for the stimulator and recording circuitry may reduce interference in stimulation signals and recorded signals especially when the stimulation module is configured to generate the stimulation signals using an ongoing stream of digital commands received from the controller.

Disclosed herein are systems and methods that relate to an integrated system that combine neural stimulation and digital logic into small modules that can be controlled with a digital interface by an external controller. According to some embodiments, systems consistent with the present disclosure may also be used with remote wireless applications for experiments with freely behaving animals or untethered human stimulation.

Functional stimulation waveforms typically consist of brief monophasic or biphasic current or voltage pulses that cause neurons around the connected electrode to generate action potentials for each stimulation cycle. Multiple electrodes can also be stimulated with grouped waveforms that use interactions between the electrodes and neurons to produce desired activation patterns. These can also include interferential patterns in which electrodes are cycled at high frequencies (>1 kHz) with slight frequency differences that produce beat stimulation frequencies in overlapping areas of current excitation. Current or voltage waveforms may have constant amplitude or may be shaped to generated desire neural recruitment. Stimulation with cyclical and pulsatile waveforms can also be used for producing neuromodulation effects in tissue.

The embodiments of the disclosure will best be understood by reference to the drawings, wherein like parts may be designated by like numerals. The components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure as claimed. Rather, the detailed description is merely representative of possible embodiments of the disclosure. In addition, the steps of a method do not necessarily need to be executed in any specific order, or even sequentially, nor need the steps be executed only once, unless otherwise specified.

Certain aspects of the embodiments disclosed herein may be implemented as software modules or components. As used herein, a software module or component may include any type of computer instruction or computer executable code located within a memory device that is operable in conjunction with appropriate hardware to implement the programmed instructions. A software module or component may, for instance, comprise one or more physical or logical blocks of computer instructions, which may be organized as a routine, program, object, component, data structure, etc. that performs one or more tasks or implements particular abstract data types.

In certain embodiments, a particular software module or component may comprise disparate instructions stored in different locations of a memory device, which together implement the described functionality of the module. Indeed, a module or component may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules or components may be located in local and/or remote memory storage devices. In addition, data being tied or rendered together in a database record may be resident in the same memory device, or across several memory devices, and may be linked together in fields of a record in a database across a network.

Embodiments may be provided as a computer program product including a non-transitory machine-readable medium having stored thereon instructions that may be used to program a computer or other electronic device to perform processes described herein. The non-transitory machine-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/machine-readable medium suitable for storing electronic instructions. In some embodiments, the computer or other electronic device may include a processing device such as a microprocessor, microcontroller, logic circuitry, or the like. The processing device may further include one or more special purpose processing devices such as an application specific interface circuit (ASIC), PAL, PLA, PLD, field programmable gate array (FPGA), or any other customizable or programmable device.

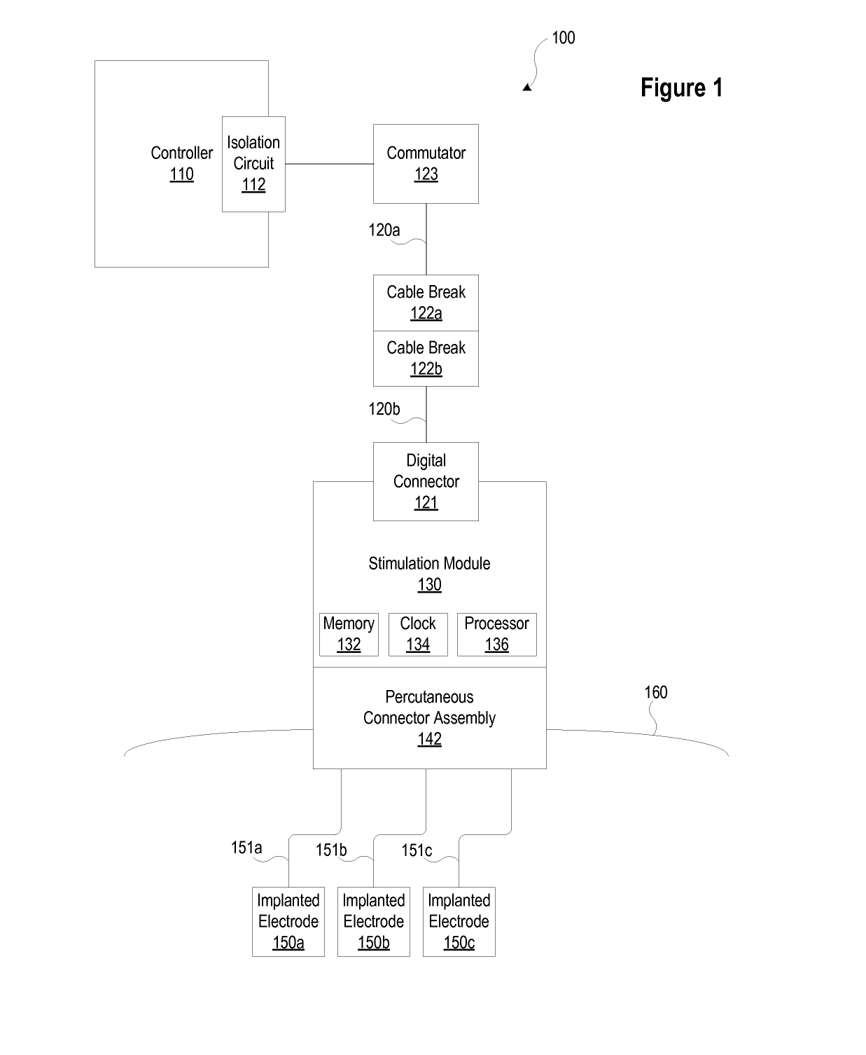

FIG. 1 illustrates a functional block diagram of a system 100 for electrical stimulation consistent with embodiments disclosed herein. Although FIG. 1 illustrates one possible implementation, many variations of system 100 are possible. A variety of embodiments are disclosed herein that incorporate numerous combinations of features. Such features may be combined in any suitable manner.

A controller 110 may be configured to control the actions of system 100. In various embodiments, the controller may comprise a computer system or other device configured to control the operation of system 100. In some embodiments, the controller 100 may comprise a system specifically designed for stimulation of neural tissue. In one specific embodiment, the controller may comprise a neural interface processor available from Ripple, LLC of Salt Lake City, Utah. In the illustrated embodiment, the controller 110 may comprise an electrical isolation circuit 112. The electrical isolation circuit 112 may utilize magnetic, capacitive or optical coupling methods to provide isolation of power and/or data in the digital interface.

In various embodiments, the controller 110 may dynamically control the stimulation module 130 with low-latency. In contrast, other embodiments, stimulation module 130 may be configured to generate a stimulation pattern in response to certain events, and then left to execute autonomously. Various embodiments of the present disclosure may provide sufficient bandwidth on the digital interface 120 to accommodate low-latency continuous control of the stimulation module 130 via the controller 110.

The controller 110 may include indicator, alert, and/or actuation devices such as: LEDs, LCD displays, audible outputs or other output devices. The controller 110 may also include methods for connecting and controlling external actuators and devices through analog and/or digital channels with SPI, RS232, I2C, CAN and other protocols. These output devices may include components for neurophysiology research such as microfluidic devices and transducers for optogenetic applications. The digital interface protocol may include methods and protocols for controlling output devices. A commutator 123 may be in communication with the controller 110. The commutator 123 may allow the cable to rotate if the subject is freely moving.

A digital interface 120 may be in communication with commutator 123. The digital interface provides transactions of digitally represented data between the system 100 and an instrumentation system (not shown) or application. System 100 may use one or more Analog to Digital Converter (ADC) elements within the module to encode analog signals into digital representations, and one or more Digital to Analog Converter elements for generation of analog signals such as DC bias and stimulation waveforms. In various embodiments, all data for stimulation control is entirely digital until such data is processed by stimulation module 130.

A cable break 122a, 122b may be provided in the digital interface 120. The cable break 122a, 122b may permit the separation between a first portion 120a and a second portion 120b of the digital interface in the event that the interface cable is pulled. The digital interface 120 may further be in electrical communication with a digital connector 121.

The digital interface 120 may be embodied as a serial or parallel connection with TTL, LVTTL, CMOS, LVDS or any other type of single-ended or differential digital signal. The digital interface 120 may include a separate clock signal for synchronizing data transfers, or it may include combined clock/data signaling such as Manchester, 8B10B coding, or others. Digital interface 120 may utilize digital signaling methods such as DC balanced codes or differential signaling to minimize common mode digital noise that can contaminate the stimulation or recording signals.

A stimulation module 130 may be in electrical communication with the digital connector 121. Although the illustrated embodiments depicts commutator 123 and cable break 122, in other embodiments, controller 110 may be directly connected to stimulation module 130.

In the illustrated embodiment, the stimulator module 130 is mechanically coupled with a percutaneous connector assembly 142 mounted on the subject 160 to minimize movement of the electrode connections within the percutaneous connector assembly 142. The percutaneous connector assembly 142 provides connections to implanted leads 151a, 151b, and 151c, which in turn are in electrical communication with implanted electrodes 150a, 150b, and 150c, respectively. In various embodiments, the implanted electrodes 150a, 150b, and 150c may represent electrodes or electrode arrays. Although FIG. 1 illustrates only one stimulation module, multiple modules can be present on a subject. Further, multiple modules may share electrode connector assemblies and digital interface cabling and connections.

The stimulation module 130 may be selectively disconnected from the percutaneous connector assembly 142. In various embodiments, the coupling between the stimulation module 130 and the percutaneous connector assembly 142 may comprise pins and sockets, zero insertion force connections, pads mated with spring pins or anisotropically conducting materials, short mechanically constrained cables and the like.

The stimulation module 130 may comprise a variety of components. In the illustrated embodiment, the stimulation module 130 comprises a memory 132, a clock 134, and a processor 136. The memory 132, which may comprise ROM, RAM, or the like, may be configured to hold sets of stimulation patterns or may hold digital commands. The memory 132 may include accessible non-volatile computer-readable memory for storage of configuration information such as: model identifiers, hardware and software revision information, hardware options, programmable options, serial numbers, manufacturing and calibration dates, calibration data for individual channels and signals, and other information. The system 100 may also include special startup and initialization modes for device discovery, bus enumeration, accessing of non-volatile info, and/or means for querying non-volatile information during operation.

The clock 134 may generate a time signal used by processor 136 or other components. The clock 134 may comprise crystals or other oscillators. Processor 136 may implement a plurality of state machines or other digital logic for generating timed patterns for each electrode. The protocol for controlling the state machine or logic may include low-level commands that allow direct synthesis of stimulation waveforms, or higher level commands that represent more complex stereotypical patterns such as: pulses, pulse pairs, pulse sets, pulse bursts, sinusoidal cycles, sine wave bursts, or other patterns.

A clock signal may be derived from timing clocks for digital interfaces, logic, state machines, and stimulation waveform generation from the digital interface, or other external clocks. In the event that the clock to the stimulator is disrupted, it is possible that the stimulator logic may hang in a state that is generating output current, the watchdog module 390 halts this output current when the loss of clock it detected. Clocks from the digital interface may be used to synchronize the actions of multiple stimulator modules.

In alternate embodiments, the clock for the stimulation module digital logic may be derived from an oscillator or other electronic clock generator contained in the stimulation module. The stimulation module digital output will run asynchronously from the controller clock or digital interface.

The stimulator of the system 100 may operate by generating controlled voltage and/or current output waveforms that are applied to electrodes. According to some embodiments, the stimulator may include analog level and/or digitally programmable range limitations for the outputs of the stimulator circuitry. According to such embodiments, a single design may be used for a wide set of output ranges for different applications, while limiting the output to reasonable levels for that application. Such limits may also help to limit the maximum currents that may be inadvertently generated by the user or by system failures. The stimulator may also include programmable ranges that are sufficiently small (e.g., below 1 μA peak-to-peak) to synthesize low-level sine waves and other signals for measurement of electrode impedance. The stimulator may also include the capability to generate DC and other waveforms for the conditioning of electrodes or lesioning tissue.

The stimulation module 130 may be selectively coupled to percutaneous connector assembly 142. The percutaneous connector assembly 142 may, in certain embodiments, be coupled to a subject in proximity to neural tissue, such as the skull of an animal or human. The percutaneous connector assembly 142 may be connected to a variety of electrodes 150a, 150b, and 150c using leads 151a, 151b, and 151c, respectively. The electrodes 150a, 150b, and 150c may be embodied in various embodiments as microelectrodes, cortical electrodes, subdural electrodes (macro or micro electrode types), spinal electrodes, intramuscular electrodes, epimysial electrodes, nerve cuff electrodes, epineurial electrodes, depth electrodes, penetrating and surface microelectrode arrays, intrafascicular electrodes, nerve cuffs or the like.

Stimulation can be for direct functional control of electrically active tissue or modulation of neural or electrophysiological activity. When stimulating with constant current, some embodiments consistent with the present disclosure may monitor the output response voltage generated for each electrode being driven. The system 100 may include a method for measuring the response voltage of each electrode during stimulation, such as a selectable amplifier that can scale the possible full-scale range of the stimulator output voltage to a range that can be digitized by an ADC. This may be a separate ADC, or an ADC that is shared and multiplexed to measure other signals within the module. The module may also use a differential amplifier for measuring the response voltage with respect to a reference electrode that is separate from the return electrode used for stimulation currents. This can help prevent overpotentials on the return electrode from corrupting measurements of the response and overpotential voltages of the stimulation electrodes. This separate reference electrode can also be used for improving the accuracy of voltages used for biasing and exhausting functions.

The resting and stimulated voltages for the electrodes can also be used to detect problems and faults with the electrodes and output circuitry of the stimulator. Stimulation response waveforms can be tested against known templates for stimulation impedance and voltage features. The system 100 may include methods for setting safety limits for these features and other basic features such as peak response voltage or total estimated charge per cycle. These limits may be used to prematurely stop or limit stimulation cycles or force the module into a fail-safe condition. These voltages can also be used to verify system calibration with calibration loads connected to the stimulator output, or calibration loads that are integrated with electronic switches into the stimulator module.

According to some embodiments, system 100 may allow for stimulation channels to be routed to separate connectors. Alternatively, a user may route stimulation channels and recording channels together. Certain embodiments may record low-level neural signals, such as extracellular Local Field Potentials (LFPs) and single/multi-unit spike signals, more macroscopic biopotential signals such as EEG, EMG, ECG, EOG, and any other type of electrophysiological signal. Still further, the neural signal amplifier may also be implemented with differential inputs for each channel, and/or with arrays of single-ended electrodes that are amplified with respect to a common reference.

Stimulation currents may create artifacts on low-level neural amplifiers. In some instances, the artifacts may temporarily saturate circuit elements (e.g., internal high-pass filters) in the amplifiers. Accordingly, certain embodiments consistent with the present disclosure may also include circuitry to quickly settle or reset the high-pass filters and other elements of the circuit that may be vulnerable to saturation. The fast settle functions may be programmable and may be applied to amplifiers connected to electrodes being stimulated and/or other electrodes that may also pick up stimulation artifacts. The fast settle function may also be used to quickly settle motion or other artifacts on the neural recording electrodes, and may be programmable to engage when the amplified neural signals reach preset limits.

Certain embodiments consistent with the present disclosure may allow for virtually simultaneous recording and stimulation from the same electrode. Such functionality may be enabled by fast settle circuitry that allows system 100 to rapidly recover from stimulation transients that saturate the neural signal amplifier. According to some embodiments, stimulation cycles for functional stimulation may be between 30 to 50 Hz at maximum (repeating with a period of 33 to 20 ms) with each stimulation pulse cycle typically lasting only 1 to 4 ms. The fast settle circuitry utilized by certain embodiments of the present disclosure may settle within 1 to 2 ms, thus leaving several milliseconds between each stimulation cycle in which reliable recordings can be obtained. This allows for a significant overall percentage of the recording to be captured. For some recorded neural signal processing methods, such energy metrics within certain frequency bands higher than the stimulation pulse frequencies, the processed recording metrics can still be captured with reasonable fidelity. Applications where this may be particularly useful include neuroprosthetic applications in which electrodes are in neural tissue with both neurons that are signaling useful information and neurons that are useful for stimulation. For example, in peripheral nerve implants for amputees, electrodes can often both record efferent activity associated with movement intent for the phantom limb and create sensations in the phantom limb when stimulated. Recorded activity in electrodes is often assessed with energy metrics that can tolerate brief interruptions in the recordings. Accordingly, it may be possible to simultaneously estimate movement intent and create sensations with the neurons around the same electrode.

Electronic circuits may be included to allow one or more neural signal amplifiers to be disconnected from an electrode. This feature may be used in conjunction with other means to avoid or recover from stimulation artifacts or in the calibration of the neural amplifiers by connecting the recording input to a calibration signal.

The power supplies for the elements of the modules may be derived from internal sources such as batteries, super capacitors, optical or infrared power recovery or other sources. In wireless applications, the power may also be provided to the module by inductive, RF, or other methods for providing power. For wired digital interfaces, the power may be provided by the same cabling used for the interface, and may include multiple supply voltages for different circuit elements, or internal power subsystems which may generate needed supply voltage(s) from the supply voltage(s) provided by the interface. These power subsystems may include linear, switching, inverting, rail-splitting, or other power supply generation circuits.

Analog circuits used for recording and analog circuits used for creating stimulation waveforms may create potentially harmful unintended currents when power supplies are partially disrupted. Accordingly, the system 100 may include switches for controlling the application of power to the analog recording and/or stimulation circuits until the power supplies provided to the module can be verified. Similarly, the system 100 may include methods for disconnecting the power supplies from the analog recording and/or stimulation circuits if the power supplies are not correct or disrupted by faulty electrical connections or other partial failures. These power supply control methods may include electronic switches, transistors, FET devices, or other power control devices to disconnect and/or shunt supplies for analog circuits to safe voltage levels. The power control methods can also be used to connect and disconnect power supplies in specific orders for analog and/or digital control circuits that require specific power supply sequences during startup and shut down. These methods may also be used for disconnection of power supplies in the event of other detected system failures such as the disruption of the digital interface, reception of invalid data, or other detected external or internal failures.

The circuit elements of the module may be implemented with combinations of discrete components (e.g., resistors, capacitors, inductors, diodes, transistors), commercial ICs (e.g., power supply, ADC, DAC, switch and other integrated devices), programmable logic (e.g., FPGAs and CPLDs), and/or custom silicon components (e.g., Application Specific Integrated Circuit or “ASIC” parts).

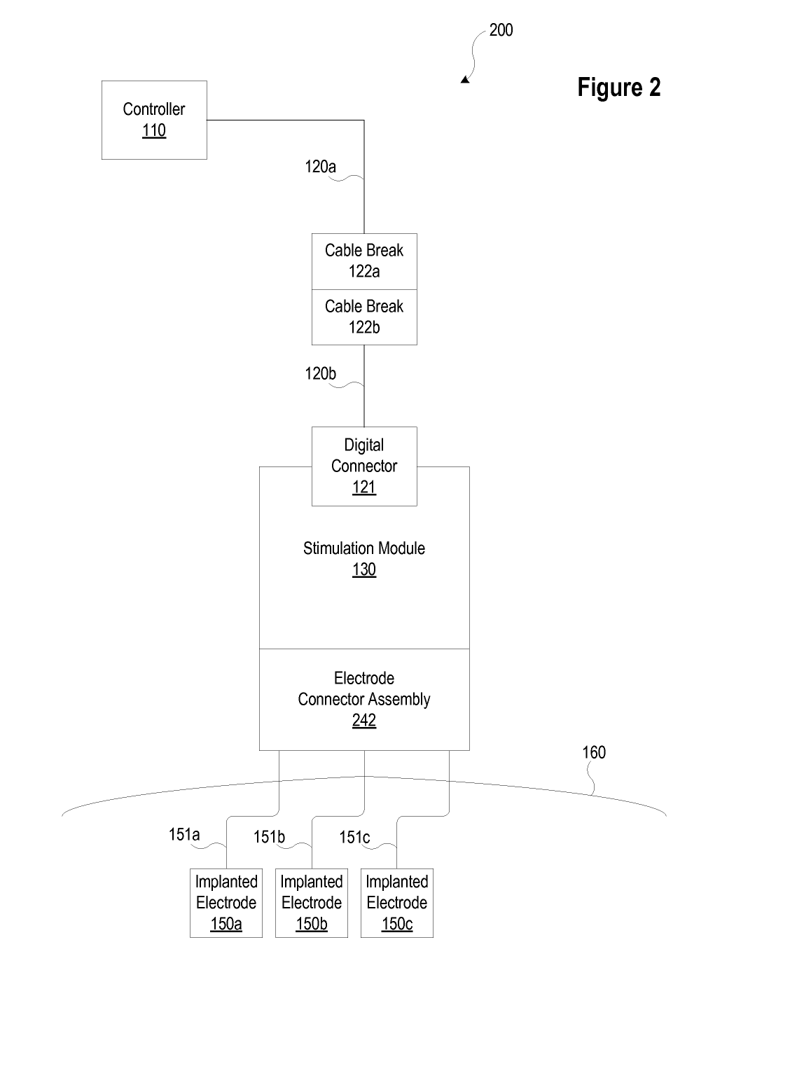

FIG. 2 illustrates a functional block diagram of a system 200 for electrical stimulation of neural tissue in which the electrode connector assembly 242 may be used to connect to electrodes 150 temporarily while the stimulation module 130 is held nearby to the subject 160 consistent with embodiments disclosed herein. A compact stimulation module 130 with a digital interface 120, as illustrated in FIG. 2, may reduce the cabling in the system 200 and, in case of recording, may also reduce noise associated with motion of the components of the system.

In some embodiments, the electrode connector assembly 242 may allow the stimulation module 130 to be selectively connected to and disconnection from electrode connector assembly 242. In other embodiments, the stimulation module 130 and the electrode connector assembly 242 may be inseparable.

In the embodiment illustrated in FIG. 2, the stimulation module 130 or electrode connector assembly 242 may be externally fixed in place near the subject, or it may be mounted to the subject as in the case of implanted electrodes with percutaneous wires and leads, as illustrated in FIG. 1. The stimulation module 130 can also be mounted in other ways to minimize the motion of the electrode leads 151, for example, incorporated in a head wrap in which the leads are also wrapped and mechanically constrained. In some embodiments, the electrode connector assembly 242 may have electrodes directly incorporated to the assembly without leads 151.

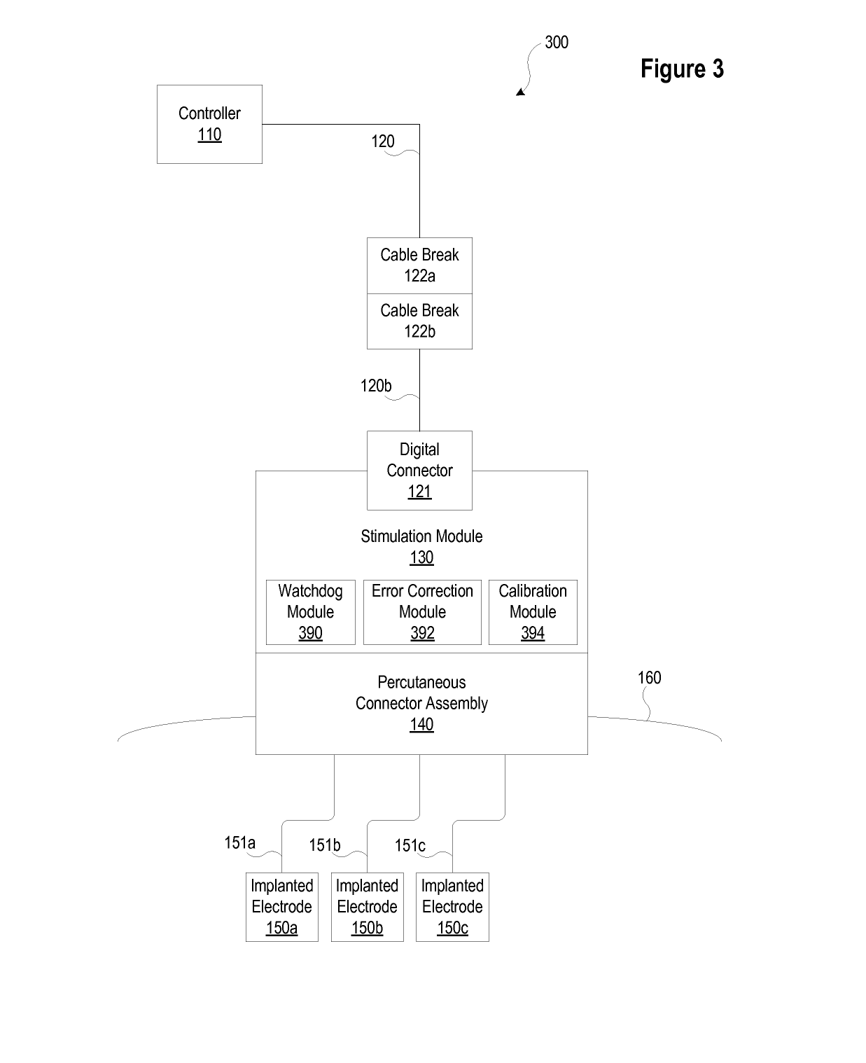

FIG. 3 illustrates a functional block diagram of a system 300 for electrical stimulation of neural tissue that includes a watchdog module 390, an error correction module 392, and a calibration module 394 consistent with embodiments disclosed herein. In order to mitigate against failure, the watchdog module 390 may issue a reset signal in the event that a clock stops for a determined period of time. The watchdog module 390 may be used to statically reset the stimulator control logic back into an off state. The reset may also be integrated so that it actuates during power up to ensure that the stimulator logic is powered into the off state.

To ensure data integrity and to prevent improper stimulation, the module may include an error correction module 392. The error correction module may apply error checking functions and/or error correction codes (e.g., parity, checksum, CRC, Hamming, or other codes), to verify sent or received digital data. The error correction module 392 may also include fail-safe modes (such as switching the stimulator output off and/or going into a safe state until reset) when erroneous or improper digital control data are received. The calibration module 394 may be configured to select a suitable level of voltage and/or current suitable for a particular subject or a particular stimulation protocol.

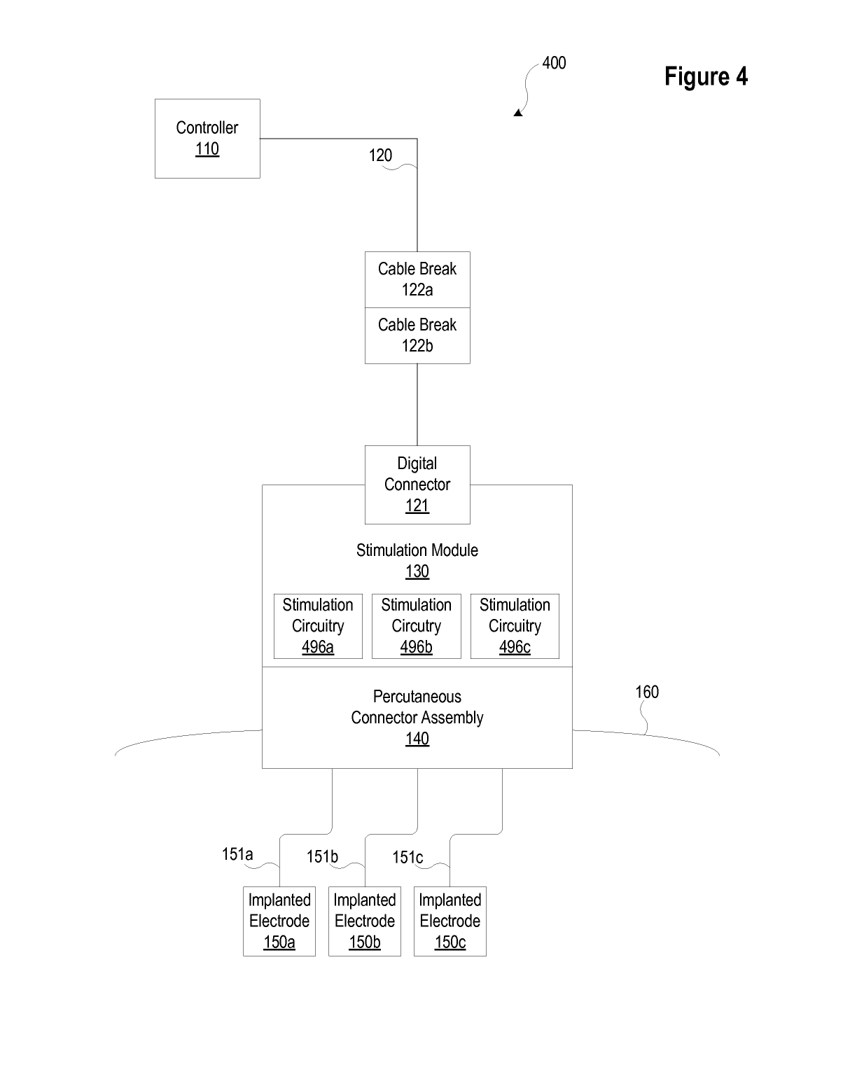

FIG. 4 illustrates a functional block diagram of a system 400 for electrical stimulation of neural tissue that includes a plurality of sets of stimulation circuitry 496a, 496b, 496c in the stimulation module 130 consistent with embodiments disclosed herein. Stimulation circuitry 496 may comprise circuits for analog or digital trimming of gain and/or voltages and/or calibrating the ADC performance and the accuracy and linearity of the stimulation circuits.

For some types of electrodes, a slight DC bias voltage may be applied through a high-value impedance when using an electrode 150 for stimulation. The DC bias voltage may drive the electrode to an overpotential that allows for higher charge injection capacity for some types of electrodes and stimulation waveforms. The stimulation circuitry 496 may include programmable DC bias values that can be applied to stimulation electrodes. According to some embodiments, the electrode's overpotential may be restored to a set DC value after each stimulation cycle or pulse sequence (commonly called “exhausting”). The system 400 may provide a current-limited switch on stimulation channels for exhausting electrodes to a set DC bias level. This exhausting current limit may be adjustable for various electrode types.

For some electrode types the total current to the electrode 150 may be DC balanced. DC balance may be enforced, according to some embodiments, by including an inline capacitor to the stimulator output. Series capacitors may also protect the electrodes from DC currents in the event of failures and faults in the stimulator circuitry. The system 400 may include series capacitors for the stimulator outputs for DC balance.

Electronic circuits may be included in the system 400 for disconnection of the electrodes from the stimulation circuitry during calibration and for connecting one or more electrode channels to a test load for calibration.

The system 400 may include programmable analog filters for processing the recorded neural signals, and digital signal processing that can apply digital filtering functions to the channel data such as offset correction, frequency filtering, and physiological indices and measures. The system 400 may also include digital processing of extracellular signals such as spike extraction and spike sorting. The raw and/or processed digital data may be transmitted over the digital interface 120 to the other components of the system.

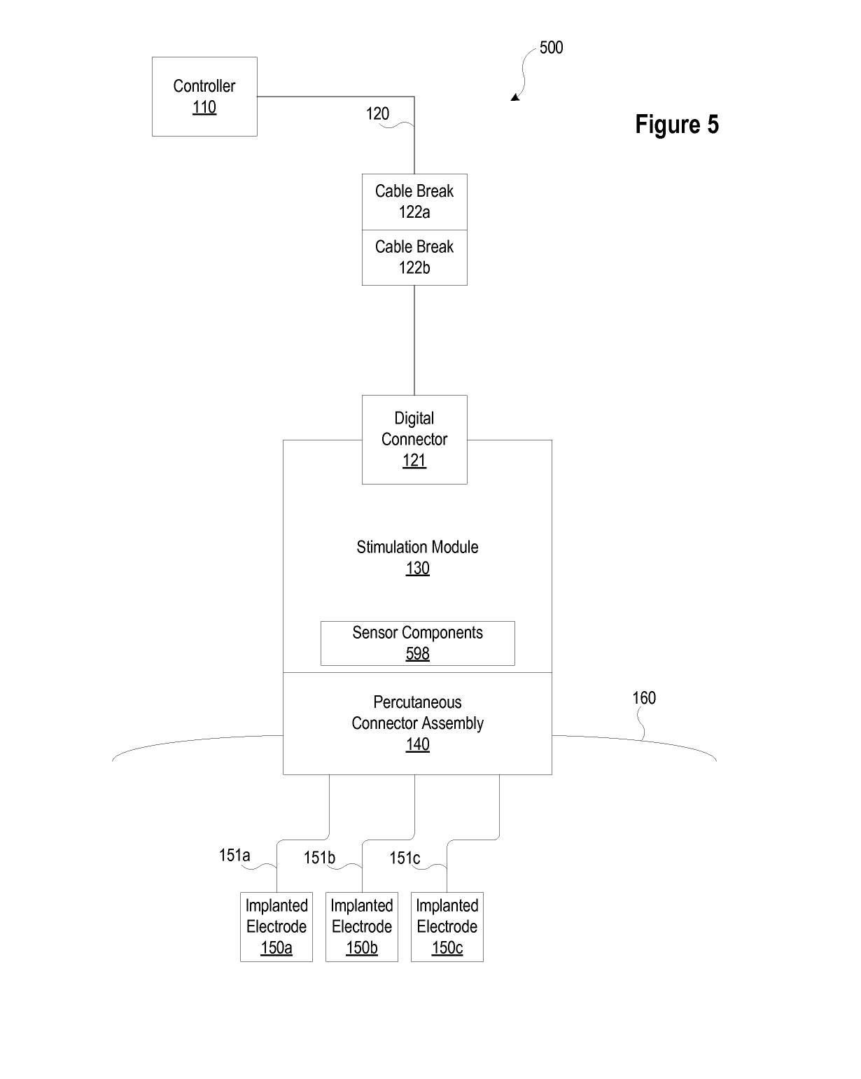

FIG. 5 illustrates a functional block diagram of a system 500 for electrical stimulation of neural tissue that includes a plurality of sensor components 598 consistent with embodiments disclosed herein. In various embodiments, the plurality of sensor components 598 may include analog and digital sensors, such as temperature measurement devices, accelerometers and gyroscopes, pressure and force sensors, voltage and current monitors, GPS units, and other devices. The system 100 may include methods for connecting and accessing other external sensors and devices through analog and/or digital channels with SPI, RS232, I2C, CAN and other protocols. The digital interface 120 may include methods and protocols for accessing sensors and input devices.

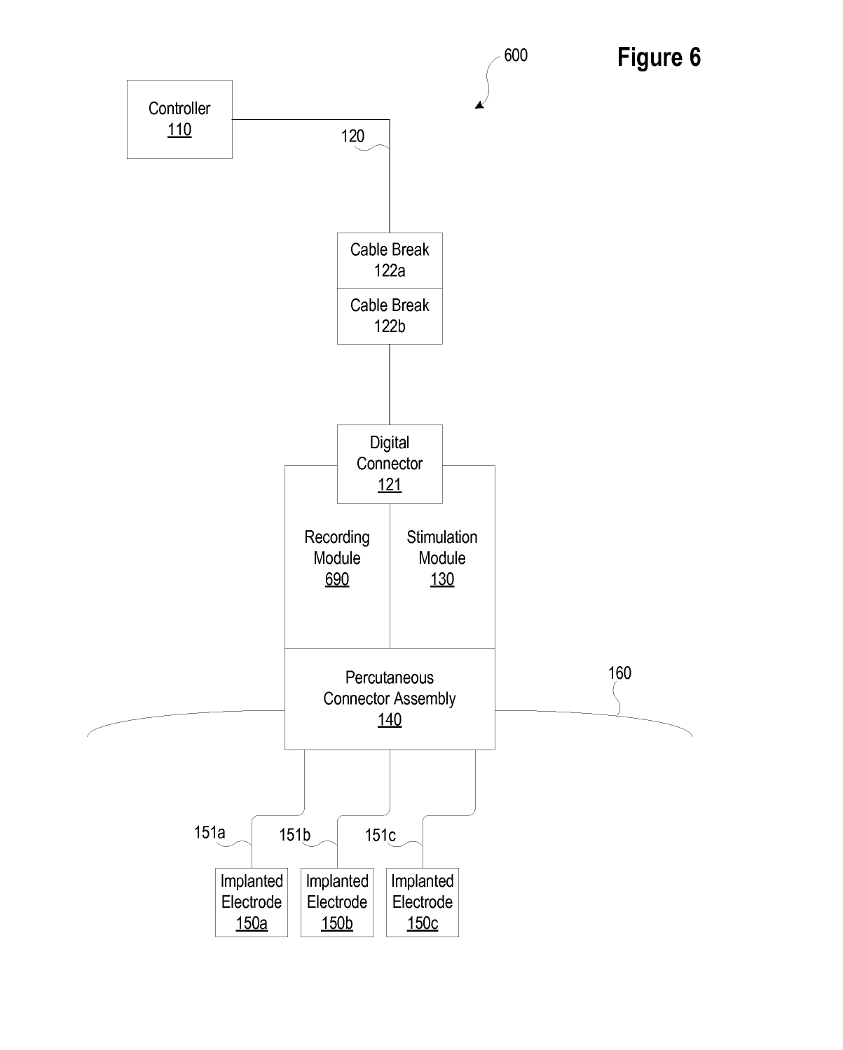

FIG. 6 illustrates a functional block diagram of a system 600 for electrical stimulation of neural tissue that includes a recording module 690 consistent with embodiments disclosed herein. In various embodiments including stimulation and recording functions, the digital interface 120 may include bidirectional paths for simultaneous exchange of stimulation and recording data. According to some embodiments, system 600 may allow for stimulation channels and neural recording channels to be routed to separate connectors.

Various techniques may be utilized in connection with system 600 to reduce noise in the recorded signals. One specific technique may involve mechanical coupling among components within system 600. Movement of components within system 600 may create capacitive microphonics that can interfere with the recording of neural signals. In some embodiments, materials may be used to dampen movements (e.g., rubber) to reduce movement being transferred to system 600.

Temporary placement of electrodes 150 may occur in various circumstances, including intraoperative use, in which an electrode is placed on exposed tissues such as the brain or temporarily implanted for cortical mapping studies. Electrodes may also be temporarily placed in subjects undergoing recording procedures, such as placement of implanted cortical electrodes for epilepsy mapping. In such procedures, electrode leads may directly exit the skin and be routed to connector assemblies placed on the head or body of the subject. In these cases, the use of a compact local stimulator with digital interface may enable the stimulator to be placed on the subject (e.g., within a head wrap) and coupled to the electrodes with reduced connections and minimal connector assemblies. In various embodiments, recording module 690 may be configured to record larger scale voltages or currents present when the simulation module 130 is active.

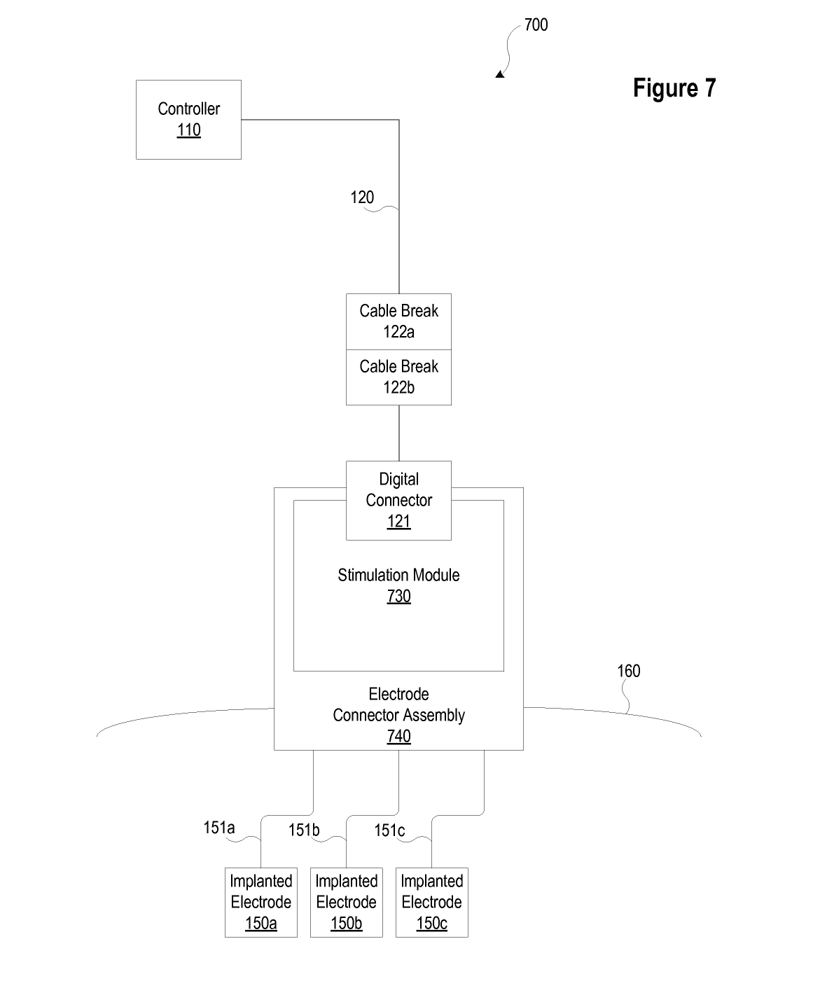

FIG. 7 illustrates a functional block diagram of a system 700 for electrical stimulation of neural tissue that includes a stimulation module 730 within an electrode connector assembly 740 consistent with embodiments disclosed herein. In the illustrated embodiment, the electrode connector assembly 740 may encompass the stimulation module 730. In the illustrated embodiment, the cable break 122a, 122b may allow for disconnection of the controller 110. In the illustrated embodiment, incorporating the stimulation module 730 directly into the electrode connector assembly 740 may reduce the number of connector contacts needed and reduce the complexity and improve the overall reliability of the implant.

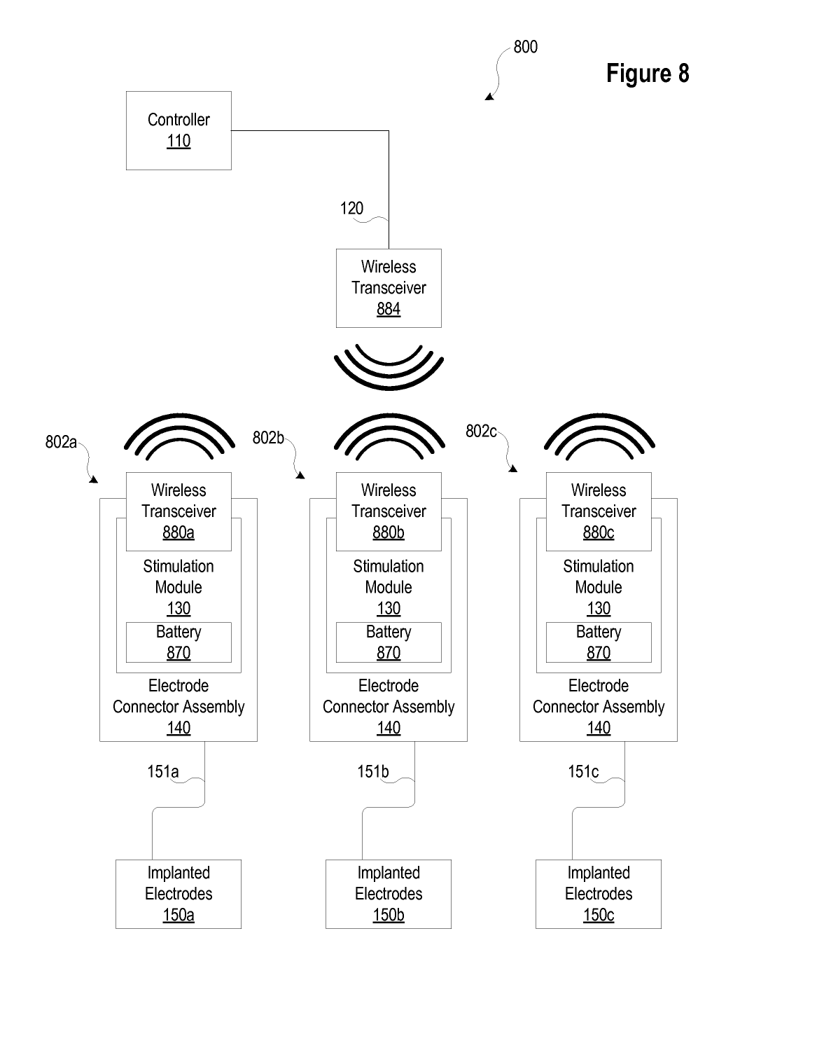

FIG. 8 illustrates a functional block diagram of a system 800 for electrical stimulation of neural tissue that includes a wireless communications between a controller 110 and a plurality of individual stimulation units 802a, 802b, 802c consistent with embodiments disclosed herein. Each of the plurality of individual stimulation units 802a, 802b, and 802c may include a wireless transceiver 880a, 880b, and 880c, respectively. A wireless transceiver 884 in communication with the controller 110 may be configured to exchange data with the wireless transceivers 880a, 880b, and 880c.

The wireless transceivers 880a, 880b, and 880c may communicate with each other and with wireless transceiver 884 using a variety of communication protocols and technologies. In various embodiments, the stimulation module 130 may comprise a battery 870 that may provide power to the stimulation module 130 and the wireless transceivers 880. Placement of the battery in the stimulation module 130 may facilitate access to the battery 870 for purposes of replacing or servicing battery 870.

In various embodiments, wireless communication among the components of system 800 may be accomplished using modulated RF technologies, such as OOK, AM, PM, FM, ODFM, or other methods. Further, such communications may utilize custom protocols or standardized protocols such as Zigbee, Bluetooth, 802.11, ultra-wide band (UWB), Bluetooth®, and other RF methods. The system 800 may also be wirelessly interfaced via infrared, visible light, or other types of radiant energy that can be exchanged across open space or through fiber optic cables.

In both wired and wireless applications, multiple instances of system 800 may be operated together across redundant digital interfaces, or over shared digital interfaces with multiplexing schemes such as Time Division Multiple Access, Code Division Multiple Access, frequency division such as different RF carrier frequencies or different wavelengths of light, or other methods for shared digital access of devices across a bus with multiple connections or wireless channel.

In both wired and wireless applications, the module may include methods for handling, adjusting, and/or compensating for multi-path RF distortion, or variable phase delay between transmitted and received signals to/from the modules. For example, the bus may include a means of adjusting the phase and timing of the digital interface acquisition clocks to accommodate varying transmission delays or different cable characteristics. In addition, the modules may use established start-of-transaction or other alignment or demarcation codes that allow the phase delay adjustment to be periodically recalibrated or tracked in real time.

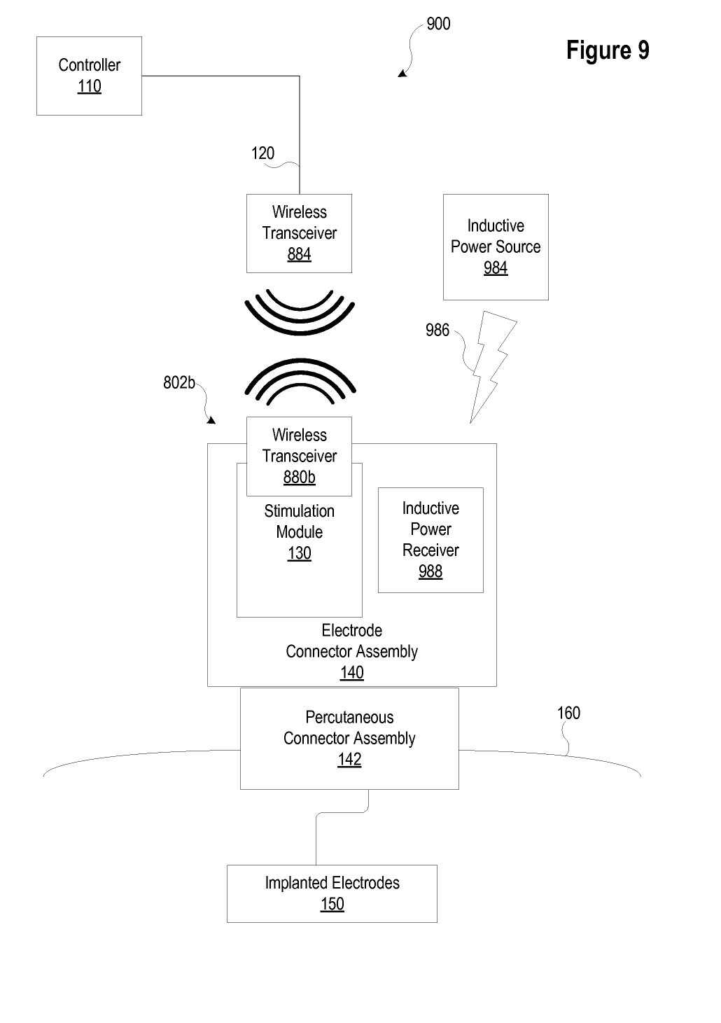

FIG. 9 illustrates a functional block diagram of a system 900 for electrical stimulation of neural tissue that includes an inductive power receiver 984 consistent with embodiments disclosed herein. An inductive power source 984 may wirelessly transmit power 986 to an inductive power receiver 988. In some embodiments, the communication link may utilize load modulation to signal digital data back to the inductive system, or other inductive coupling methods.

Multiple devices may be synchronized to timing clocks in the digital interface or other external broadcast clocks (such as RF, Infrared Light or other methods).

In various embodiments, stimulators may be configured to generate short bursts of repeating patterns and to execute pre-loaded patterns in response to specific external events. In some embodiments it may be desirable to dynamically control a stimulator with control data streamed in real time from an external controller. This can include cases where the stimulation pattern is too complex to pre-load the control information into the stimulator for execution independent of the controller, and also cases where the stimulation must be rapidly configured or changed in complex ways in response to real time events such as environment changes, behavioral cues, physiological conditions, treatment protocols, experimental demands, etc.

The signals carried by electrodes can be very with high source impedances and susceptible to noise contamination. It is therefore desirable to keep the cabling and connectors between the stimulator circuitry and the electrodes as short as possible to minimize external interference that can couple to these cables. It is also desirable to keep the cabling and connectors between the stimulator and electrodes from moving to prevent generation of movement-related noise and artifacts. These are especially true when the electrodes are being used for recording simultaneously with stimulation.

In various embodiments, the stimulator may be embedded into a module that can be mounted to a percutaneous connector assembly, held close to the body with the stimulator directly connected to the electrodes (e.g. intraoperative use), or mounted to the body, especially such that the stimulator and electrode connection leads are mechanically fixed to prevent motion artifacts. The stimulator may be controlled with digital control data that can be preloaded for execution or continuously streamed in real time from an external controller. In contrast with other systems, embodiments of the present disclosure may use completely digital interfaces for both control of the stimulator, and communication of recorded activity and other signals back from the module to the controller or other external devices. In still other embodiments, a variety of features may be included, including but not limited to:
Calibration of stimulation outputs;
Switching of stimulation output to calibration load;
Disconnecting stimulation outputs from an electrode connection;
A circuit to return electrode to pre-stimulation voltage in a current controlled manner, commonly called exhausting;
Variable exhausting current levels;
Monitoring or recording output voltage caused by a stimulation pulse;
Series capacitor(s) between the stimulation generator and electrode connection to ensure charge balance of pulses and/or to block DC currents due to a circuit fault;
Power supply monitoring for safety;
Stimulation module shut-down sequencing;
Detecting inappropriate voltage response to a stimulation pulse and halting stimulation;
Limiting the stimulation amplitude (voltage or current) for particular applications;
Providing a separate reference from the stimulation current return path;
Single-ended recording of neural/muscle (electrophysiological) signals with shared reference or differential inputs;
Programmable analog filters;
Electrophysiological high pass filters/amplifiers having fast settling times;
Ectrophysiological amplifiers with automatic fast settling times;
Same-electrode rapid recovery from stimulation artifact;
Exhausting as part of fast settle function;
A separate reference from the stimulation current return path;
disconnecting electrophysiological amplifiers from the electrode interface for testing or protection from stimulation artifacts;
Digital signal processing techniques including:
   signal filtering,
   spike extraction (threshold),
   spike sorting, and/or
   signal energy (EMG);
Simultaneous use of multiple stimulators modules controlled by one controller with synchronized clocks for multiple devices (on wired digital interface or other broadcast clock);
Electrode impedance measurement capability (low level stimulation current);
Memory for storing configuration and/or calibration data
Optical (LED) outputs as indicators or for motion tracking;
Optical outputs for optogenetic stimulation;
Other sensors such as GPS, Humidity, Temperature, etc.;
I2C, SPI interfaces for exchanging data with sensors;
Battery to power stimulator
Internal clock in stimulator for driving stimulation digital logic and/or state machines;
Memory for storing pre-programmed stimulation or buffered, complex, stimulation patterns; and
DC balanced digital signaling protocol, such as 8b10b or Manchester encoding, to minimize digital interference to analog circuitry

1. An electrical stimulation system configured to stimulate tissue in a subject, the system comprising:
a controller configured to send at least one stimulation pattern to be implemented by the electrical stimulation system, the controller comprising:
a first digital control interface;
a stimulation module, comprising:
a second digital control interface configured to be in electrical communication with the first digital control interface;
stimulation circuitry configured to implement the at least one stimulation pattern as an analog stimulation signal based on an ongoing stream of digital commands received from the controller; and
a first connector;
a percutaneous connector assembly configured to be coupled to a subject through the subject's skin, the percutaneous connector comprising:
a second connector configured to couple to the first connector; and
a first electrode lead configured to be in electrical communication with a first implanted electrode and the second connector;
wherein the stimulation module may be selectively in electrical communication with the controller.

2. An electrical stimulation system configured to stimulate tissue in a subject, the system comprising:
a controller configured to send at least one stimulation pattern to be implemented by the electrical stimulation system, the controller comprising:
a first digital control interface;
a stimulation module, comprising:
a second digital control interface configured to be in electrical communication with the first digital control interface;
stimulation circuitry configured to implement the at least one stimulation pattern as an analog stimulation signal based on an ongoing stream of digital commands received from the controller; and
a first connector;
an electrode connector assembly configured to be disposed external to the subject's skin, the electrode connector assembly comprising:
a second connector configured to couple to the first connector; and
a first electrode lead configured to be in electrical communication with a first implanted electrode;
wherein the first electrode lead is configured to pass through the subject's skin and transmit the analog stimulation signal to an electrode implanted in the subject;
wherein the stimulation module may be selectively in electrical communication with the controller.

3. An electrical stimulation system configured to stimulate tissue in a subject, the system comprising:
a controller configured to send at least one stimulation pattern to be implemented by the electrical stimulation system, the controller comprising:
a first digital control interface;
a percutaneous connector assembly configured to be coupled to the subject through the subject's skin, the percutaneous connector comprising:
a stimulation module, comprising:
a second digital control interface configured to be in electrical communication with the first digital control interface;
stimulation circuitry configured to implement the at least one stimulation pattern as an analog stimulation signal based on an ongoing stream of digital commands received from the controller;
a first electrode lead configured to be in electrical communication with a first implanted electrode.
wherein the stimulation module may be selectively in electrical communication with to the controller.

4. The electrical stimulation system of claim 1, 2, 3, or 4, further comprising:
a recording module, comprising:
amplification and digitization circuitry configured to generate a digital representation of a neural signal;
wherein the first digital control interface is further configured to permit communication of the neural signal to the controller via the first digital control interface.

5. The electrical stimulation system of claim 4, wherein the neural signal is received via one of the first implanted electrode and a second implanted electrode configured specifically to record neural signals.

6. The electrical stimulation system of claim 1, 2, 3, or 4, wherein the subject comprises one of an animal and a human.

7. The electrical stimulation system of claim 1, 2, 3, or 4, wherein the controller is further configured to proximately change at least one stimulation parameter of the stimulation and while the controller is coupled to the stimulation module.

8. The electrical stimulation system of claim 1, 2, 3, or 4, wherein the stimulation module is configured to selectively generate the analog stimulation signal while the controller is out of electrical communication with the stimulation module.

9. The electrical stimulation system of claim 8, wherein the analog stimulation signal is generated in response to at least one of passage of a time interval, an external event, and an electrophysiological event.

10. The electrical stimulation system of claim 1, 2, 3, or 4, wherein the first digital control interface and the second digital control interface each comprise a wireless data transceiver.

11. The electrical stimulation system of claim 10, wherein the wireless data transceiver is configured to communicate using a data protocol selected from the group consisting of Zigbee, Bluetooth, and IEEE 802.11.

12. The electrical stimulation system of claim 10, wherein the wireless data transceiver comprises one of a radio frequency transceiver, an infrared transceiver, and a visible light transceiver.

13. The electrical stimulation system of claim 10, further comprising:
an indicative power source configured to generate an electromagnetic field;
wherein the stimulation module further comprises an inductive power receiver configured to convert the electromagnetic field into electrical power for use by the stimulation module.

14. The electrical stimulation system of claim 1, 2, 3, or 4, further comprising electrical isolation circuitry located between the controller and the stimulation module.

15. The electrical stimulation system of claim 1, 2, 3, or 4, wherein one of the first digital control interface and the second digital control interface comprises an adjustable phase data clock to account for a transmission delay.

16. The electrical stimulation system of claim 1, 2, 3, or 4, wherein the one of the first digital control interface and the second digital control interface are configured to utilize differential signaling to reduce common mode signals and interference.

17. The electrical stimulation system of claim 1, 2, 3, or 4, wherein the stimulation module further comprises:
an error correction module configured to verify a digital communication received via the second digital control interface and to discontinue generation of the analog stimulation signal upon failing to verify the digital communication.

18. The electrical stimulation system of claim 17, wherein the error correction module is further configured to verify the digital communication using one of a parity check, a check sum, a cyclical redundancy check, and a Hamming Code.

19. The electrical stimulation system of claim 17, wherein the error correction module is further configured to utilize one of digital communication start codes, digital communication transaction codes, and alignment codes to verify data packet integrity

20. The electrical stimulation system of claim 1, 2, 3, or 4, wherein the stimulation module further comprises:
a watchdog module configured to monitor a clock signal received via the second digital control interface and to discontinue generation of the analog stimulation signal upon detection of an inaccurate clock signal.

21. The electrical stimulation system of claim 1, 2, or, 3, wherein the stimulation circuitry comprises a plurality of distinct stimulation units configured to generate a corresponding plurality of distinct stimulation patterns.

22. The electrical stimulation system of claim 1, 2, 3, or 4 wherein the stimulation module is further configured to generate a bias voltage to apply to the first implanted electrode to increase the range of a stimulation current used to generate the stimulation pattern while performing reversible reactions between the electrode and the body.
